(19) United States
(12) Patent Application Publication
(10) Pub. No.: US 2026/0117179 A1
(43) Pub. Date: Apr. 30, 2026

(54) METHOD FOR PREPARING KILLER IMMUNOCYTES AND ITS APPLICATION IN LUNG CANCER

(71) Applicant: BINHUI BIOPHARMACEUTICAL CO., LTD., WUHAN (CN)

(72) Inventors: BINLEI LIU, WUHAN (CN); YANG WANG, WUHAN (CN); HAN HU, WUHAN (CN); JING JIN, WUHAN (CN); RUNYANG WANG, WUHAN (CN); QIN ZHOU, WUHAN (CN)

(21) Appl. No.: 19/008,938

(22) Filed: Jan. 3, 2025

Related U.S. Application Data
(63) Continuation-in-part of application No. 17/134,209, filed on Dec. 25, 2020.
Application No. 17/134,209 is a continuation of application No. PCT/CN2019/093028, filed on Jun. 26, 2019.

(30) Foreign Application Priority Data
Jun. 28, 2018 (CN) 201810706038.9

(51) Int. Cl. C12N 5/0783; A61K 35/17; A61P 35/00

(52) U.S. Cl. CPC C12N 5/0638; A61K 35/17; A61P 35/00; C12N 2502/70

(57) ABSTRACT

The present disclosure a method for preparing killer immunocytes and its application in lung cancer. Autologous immunocytes of the cancer patient can be activated in vitro by the VAK technique, and by re-infusing these activated immunocytes into the patient's body, such that the immunocytes achieve a powerful tumor killing effect, especially in the secondary activation technology of immunocytes and viruses mixed twice, which can make immunocytes maintain a higher killing activity. VAK cells kill the cancer cells and meanwhile promote release of tumor associated antigen, being beneficial to inducing a specific anti-tumor immunological reaction.

### CROSS-REFERENCE TO RELATED APPLICATIONS

This application is a Continuation-in-Part Application of my application U.S. Ser. No. 17/134,209 filed on Dec. 25, 2020, which is a Continuation-Application of NO. PCT/CN2019/093028 filed on Jun. 26, 2019. This PCT also claims priority foreign priority of Chinese Patent Application No. 201810706038.9, filed on Jun. 28, 2018, the entire content of which is incorporated herein by reference.

### FIELD

The present disclosure relates to a method for preparing killer immunocytes and its application in lung cancer.

### BACKGROUND

In the prior art, multiple methods have been developed for cancer therapy, such as surgical excision, radiotherapy, chemotherapy, anticancer drugs, intratumoral injection of virus and the like, but because the cancer patients have low autoimmune function or are tolerant to cancer cells, these methods sometimes have poor effects. Therefore, during the cancer therapy, enhancing patient's own immunity and activating immunological reaction against the cancer cells are very critical.

### SUMMARY

The purpose of the present disclosure is to provide a method for preparing killer immunocytes and its application in lung cancer by VAK technique, cancer cells can be killed and meanwhile the release of tumor associated antigen is promoted, being beneficial to inducing a specific anti-tumor immunological reaction.

In order to realize the above purpose, the method for preparing killer immunocytes, comprises the following steps:
1) isolating immunocytes from malignant pleural effusion samples of a subject;
2) co-incubating Ultra-Violet-inactivated herpes simplex virus type II with the immunocytes to activate the immunocytes;
3) removing the Ultra-Violet-inactivated herpes simplex virus type II to obtain the activated immunocytes;
4) add new Ultra-Violet-inactivated herpes simplex virus type II to the activated immunocytes for secondary activation, thereby obtaining killer immunocytes for backinfusion into the subject's pleural fluid.

Optionally, in step 2) of the above method, a time of the co-incubating is 36-48 hour.

The invention also discloses a method for treating lung cancer, comprising administering an effective amount of the killer immunocytes prepared by the above method to a subject, and infusing more than 10⁶ killer immunocytes back into the subject's pleural fluid at a time.

In the method for treating lung cancer, a frequency of infusing is once every 4-9 days, with 3-4 times per course of treatment, and the subject is given 1-2 courses of treatment. Optionally, a frequency of infusing is once a week, with 3 times per course of treatment, and the subject is given 1-2 courses of treatment.

Preferably, the preservation number of Ultra-Violet-inactivated herpes simplex virus type II is CGMCC No. 3600.

The method provided by the present disclosure adopts the VAK (Virus activated killer) technique, which is the inventors' innovative technique, and the virus-activated immunocytes are re-infused into the body of the patient, thereby achieving the anti-tumor effect. Some cancer patients have low immunocytes function or are tolerant to the cancer cells, the virus can effectively activate these immunocytes (the immunocytes activated by the VAK technique are called VAK cells for short), such that the immunocytes achieve a powerful tumor killing effect, especially in the secondary activation technology of immunocytes and viruses mixed twice, which can make immunocytes maintain a higher killing activity. VAK cells kill the cancer cells and meanwhile promote release of tumor associated antigen, being beneficial to inducing a specific anti-tumor immunological reaction.

The method provided by the present disclosure has the beneficial effects: autologous immunocytes of the cancer patient can be activated in vitro by the VAK technique, and by re-infusing these activated immunocytes into the patient's body, a good anti-tumor effect can be achieved, and because these immunocytes are autologous immunocytes, there is no rejection, being safe and reliable.

### BRIEF DESCRIPTION OF THE DRAWINGS

FIG. 1 shows a mouse tumor size increase trend chart of in-vivo killing CT26 by UV-oHSV2 stimulated mouse PBMC (on day 17).

FIG. 2 shows a mouse tumor size increase trend chart of the UV-oHSV2 stimulated mouse PBMC group after reinjection of CT26 (on day 40).

FIG. 3 shows a mouse tumor size increase trend chart of in-vivo killing 4T1 by UV-oHSV2 stimulated mouse PBMC (at 40 day).

FIG. 4 shows an effect comparison diagram of in-vitro killing BGC823 by PBMC cells of the volunteer GQX (all experiment groups).

FIG. 5 shows an effect comparison diagram of in-vitro killing BGC823 by PBMC cells of the volunteer KZH (all experiment groups).

FIG. 6 shows an effect comparison diagram of in-vitro killing BGC823 by PBMC cells of the volunteer LBL (all experiment groups).

FIG. 7 shows an effect comparison diagram of in-vitro killing BGC823 by PBMC cells of the volunteer SXT (all experiment groups).

FIG. 8 shows an effect comparison diagram of in-vivo killing of LoVo by the UV-oHSV2 stimulated human PBMC (all experiment groups).

FIG. 9 shows Effusion progression free survival of MPE with VAK treatment.

FIG. 10 shows overall survival of MPE with VAK treatment.

FIG. 11 shows CT images of changes in pleural effusion in patient S001.

FIG. 12 shows CT images of changes in pleural effusion in patient 01-002.

FIG. 13 shows effect comparison of VAK between multiple groups of lymphocytes.

### DETAILED DESCRIPTION OF THE EMBODIMENTS

The disclosure is further described in detail below, in conjunction with specific examples and accompanying drawings.

The virus involved in the experiment of this example is herpes simplex virus, and was inactivated. Such herpes simplex virus includes recombinant herpes simplex virus type I, recombinant herpes simplex virus type II, wild-type herpes simplex virus type I, and wild-type herpes simplex virus type II, as follows:

oHSV1: recombinant herpes simplex virus type I, its microbial preservation number is CGMCC No. 6397, and ICP34.5 gene and ICP47 gene are knocked out in the herpes simplex virus (disclosed in authorized China patent application CN201210337627.7 “Recombinant herpes simplex virus, preparation method and application thereof”)

oHSV2: recombinant herpes simplex virus type II, its preservation number is CGMCC No. 3600. For the preserved biomaterial H2d3d4-hGF strain, the meaning of its strain number is as follows: H2 refers to herpes simplex virus type II HG52 strain (HSV2); d3 refers to a strain in which ICP34.5 is knocked out; d4 refers to a strain in which ICP47 is knocked out; hGF refers to an expression cassette in which human granulocyte-macrophage colony-stimulating factor (hGM-CSF) is inserted (disclosed in authorized China patent application CN201010116275.3 “Vector of recombinant herpes simplex virus type II and preparation method, recombinant virus, pharmaceutical composition and application thereof”).

HSV1: wild-type herpes simplex virus type I, Catalogue No. 0104151v; the virus is purchased from UK National Collection of Pathogenic Viruses (NCPV).

HSV2: wild-type herpes simplex virus type II, Catalogue No. 0104152v; the virus is purchased from UK National Collection of Pathogenic Viruses (NCPV).

### Part I: Preparation of VAK

### Embodiment 1. Preparation of Mouse VAK (Immune Cells Come from Mouse PBMC)

1.1 Collection of peripheral blood: the mouse was taken out, and blood was collected from the eyeball.
1.2. Mouse PBMC was isolated from the mouse blood by using a commercially available kit (a mouse peripheral blood lymphoid isolation kit from Tian Jin HaoYang Biological Manufacture Co., Ltd).
1.3. Preparation of mouse VAK
1) The mouse blood in a heparin sodium anticoagulation tube was put into a clean centrifugal tube, an equal volume of mouse blood sample diluent was added, and the mixture was slowly blown and beaten to be even, ready for use.
2) To a lymphocyte isolation tube of 15 mL, an equal blood volume of mouse peripheral blood lymphocyte isolation medium was added, letting the lymphocyte isolation medium fall into the bottom of a partition plate, and if the lymphocyte isolation medium could not fall into the bottom, centrifugal operation was conducted. The diluted blood sample was slowly added along the tube wall, and centrifuged by using a medical centrifugal machine 820 at 20° C. for 25 minutes.
3) An uppermost serum layer was taken out and put in a clean centrifugal tube of 15 mL, placed in a water bath at 56° C., inactivated for 30 minutes, and centrifuged under 3500 rpm at 20° C. for 10 minutes, the precipitate was discarded, and the supernatant was left, ready for use.
4) A middle milk-white lymphocyte layer was put in a clean centrifugal tube of 15 mL, and an equal volume of PBS was added, and mixed well with the lymphocyte layer, the mixture was centrifuged under 820 g at 20° C. for 10 minutes, the supernatant was discarded, and the precipitate was left.
5) The cell precipitate obtained in 4) was washed, centrifuged under 820 g at 20° C. for 10 minutes, the supernatant was discarded, and the precipitate was left. The operation was repeatedly conducted for one time.
6) The finally obtained precipitate was re-suspended with 4 mL of serum-free medium.
7) oHSV2 was sampled and put in an EP tube, and processed for 30 minutes by ultraviolet irradiation for inactivation, ready for use.
8) 100 μL of cell suspension was added in 900 μL of PBS, and counted by using a blood counting chamber.
9) The collected cells were co-incubated with oHSV2 with MOI=1, 0.1 and 0.01. The set groups are respectively a blank control group (PBMC), a solvent control group (PBMC and a stabilizer), a positive control group (PBMC and PHA) as well as stimulation groups with MOI=1, 0.1 and 0.01, totally six groups.
10) A pre-mixed suspension was placed in a six-well plate, and incubated in a CO₂ incubator.

### Embodiment 2. Preparation of Human VAK (Immune Cells Come from Human PBMC)

When the blood was collected, the volunteer was required to be in good health and in normal condition, without inflammation. One day before the blood collection, the volunteer was required to eat a light diet, without drinking wine, and to ensure sufficient sleep. At the time of blood collection in the morning of next day, the stomach was empty.

### 2.1. Processing of Blood Sample

1) Human PBMC was isolated from human blood by using a commercially available kit (Human peripheral blood lymphoid isolation kit from TianJin HaoYang Biological Manufacture Co., Ltd).
2) The finally obtained PBMC cell precipitate was re-suspended by 4 mL of serum-free medium DMEM/F12.
3) oHSV2 was sampled and put into an EP tube, processed for 30 minutes by ultraviolet radiation, and inactivated, ready for use.
4) 100 μL of cell suspension was added in 900 μL of PBS, and counted by a blood counting chamber.
5) The collected cells were co-incubated with oHSV2 with MOI=1, 0.1 and 0.01. The set groups are respectively a blank group control group (PBMC), a solvent control group (PBMC and a stabilizer), a positive control group (PBMC and PHA) and stimulation groups with MOI=1, 0.1 and 0.01, totally six groups.
6) A pre-mixed suspension was placed into a six-well plate, and incubated in a CO₂ incubator.

### 2.2. Related Information of VAK Volunteers

Related information of the volunteers for VAK preparation is seen in the following Table 1.

TABLE 1
Related information of the volunteer for blood collection

| Donor | Gender | Age |
|---|---|---|
| LBL | Man | 55 |
| LXX | Woman | 37 |
| LHP | Woman | 42 |
| CLK | Man | 25 |
| JJ | Woman | 25 |
| WRY | Man | 23 |
| GOX | Man | 40 |
| SXT | Man | 30 |
| KZH | Man | 26 |
| ST | Man | 45 |

A grouping experiment was conducted as follows for the above-described volunteers, each group was divided into 6 items, including a PBMC control group, a buffer control group, and three groups with decreased virus addition volume, and a PHA control group, the following Table 2 is a grouping experiment table for a volunteer LBL, the grouping experiment table of other volunteers is similar to this table, only the addition amount of the reagent and virus were adjusted in proportion according to the difference in number of the volunteer cell suspension.

TABLE 2
Related parameters in VAK preparation (Donor: LBL)

| Groups | PBMC | | Buffer | | MOI = 1 | | MOI = 0.1 | | MOI = 0.01 | | PHA | |
|---|---|---|---|---|---|---|---|---|---|---|---|---|
| Volume of cell | 500 | μL | 500 | μL | 500 | μL | 500 | μL | 500 | μL | 500 | μL |
| suspension | | | | | | | | | | | | |
| Volume of | 250 | μL | 250 | μL | 250 | μL | 250 | μL | 250 | μL | 250 | μL |
| autologous | | | | | | | | | | | | |
| plasma | | | | | | | | | | | | |
| 100 × diabody | 25 | μL | 25 | μL | 25 | μL | 25 | μL | 25 | μL | 25 | μL |
| Volume of buffer | 0 | | 360 | μL | 0 | | 0 | | 0 | | 0 | |
| Volume of virus | 0 | | 0 | | 360 | μL | 36 | μL | 3.6 | μL | 0 | |
| PHA | 0 | | 0 | | 0 | | 0 | | 0 | | 7.5 | μL |
| Serum-free | 1725 | μL | 1365 | μL | 1365 | μL | 1690 | μL | 1725 | μL | 1720 | μL |

medium

### Embodiment 3: Preparation of Human VAK (Immune Cells Come from Malignant Human Pleural Effusion, Activate Immune Cells Twice)

The following steps (1-2) describe the enrollment of cancer patients and the collection of pleural effusion and blood; (3) outlines the preparation process of UV-OH2; (4) explains the preparation of autologous plasma from the patient's blood; (5) details the production process of VAK cells.
(1) Patients enrolling following informed consent (conducted in the hospital): Cancer patients must undergo screening to meet inclusion and exclusion criteria and sign an informed consent form before beginning VAK treatment.
(2) Collect blood and pleural effusion: Approximately 5-10 mL of venous blood is collected for autologous plasma preparation. Not less than 100 mL of pleural effusion is collected for VAK cell preparation. These materials are then transported to the GMP facility under time and temperature-controlled conditions.
(3) Preparation of UV-OH2: The OH2 virus vial is thawed at room temperature. In a biosafety cabinet, the virus is transferred from the vial into a culture dish. It is then exposed to a UV lamp (1200 mw/m²; 254 nm) at a distance of 70-80 cm from the light source for 30 minutes.
(4) Preparation of Autologous Plasma: Transfer 5-10 mL of whole blood into a centrifuge tube, centrifuge at 1500 g for 15 minutes, and collect the upper plasma layer. Transfer this clarified autologous plasma to a new centrifuge tube, incubate at 56° C. for 30 minutes, and then centrifuge at 1500 g to obtain clear autologous plasma.
(5) VAK Cell Production Process (Isolation, Culture, Resuspend):
1) Add the patient's pleural to a 50 mL centrifuge tube, centrifuge at 500 g for 15 minutes, and discard the supernatant.
2) Re-suspend the pellet with 40 mL of sodium chloride injection (0.9%), pass through a 70 μm cell strainer, and wash the cell strainer with 5 mL of sodium chloride injection (0.9%).
3) Discard the cell strainer, centrifuge the filtered cell suspension at 400 g for 5 minutes. Discard the supernatant after centrifugation, re-suspend the pellet with 5-10 mL of sodium chloride injection (0.9%), count the cells, and dilute the cell density to approximately 10\hat{ }7 cells/mL.
4) Prior to use, mix Ficoll thoroughly and add an equal volume of Ficoll to a new 15 mL centrifuge tube.
5) Carefully overlay the cell suspension on top of the Ficoll layer.
6) Centrifuge at 400 g for 30 minutes at 18-20° C., ensuring slow acceleration and deceleration with the emergency brake setting turned off.
7) Collect the intermediate buffy coat layer (lymphocytes) into a new centrifuge tube.
8) Add 20-40 mL of sodium chloride injection (0.9%) to the collected lymphocytes, re-suspend the cells, and centrifuge at 400 g for 10 minutes at 18-20° C.
9) Repeat the washing step once, and finally, re-suspend the pellet in 5 mL of serum-free culture medium (RPMI).
10) Take 20 μL of cell suspension in an EP tube, add an equal volume of trypan blue stain, gently pipette, and count using a cell counter.
11) Maintain the cell culture density within the range of 10\hat{ }6 during seeding of immune cells (RPMI). Then, appropriate amounts of levofloxacin and autologous plasma were added to the cell suspension and mixed well.
12) Inoculate the cell suspension with inactivated virus (UV-OH2, multiplicity of infection MOI=1) to activate immune cells. The amount of UV-OH2 added is calculated based on the titer of OH2 before inactivation.
13) After 36-48 hours of culture, centrifuge the cell suspension at 400 g for 10 minutes, discard the supernatant, and add 30 mL of sodium chloride injection (0.9%) to wash the cells and collect the cell pellet.
14) Repeat the washing step, centrifuge at 400 g for 10 minutes, and discard the supernatant.
15) Resuspend the lymphocytes with 10 ml of sodium chloride injection (0.9%) into a 50 ml centrifuge tube (DS).
16) Add UV-OH2 (multiplicity of infection MOI=1) to the DS of VAK and mix well. Collect 20 μL of the cell suspension for cell count determination. Replenish the cell suspension to 50 mL with sodium chloride injection (0.9%), and transfer it into a single-use plastic blood bag. Finally, seal the bag (DP).
17) DP was transported to the hospital under temperature and time conditions and infused into the patient's pleural cavity.
18) Assess pleural effusion volume (WHO) and evaluate target lesions (RECIST1.1) 28 days after the end of treatment.

### Part II. VAK Effect Verification

### Experimental Example 1. In-Vivo Killing of CT26 Tumor Cells by UV-oHSV2 Stimulated Mouse PBMC

### 1.1. Preparation of CT26 Cells

Mouse colon cancer cells CT26 were cultivated according to the conventional method, and the medium was DMEM/F12 containing 10% newborn bovine serum. Before tumor induction, the cells were collected, and centrifuged under 2800 rpm for 3 minutes, finally the cell precipitate was re-suspended with a serum-free DMEM/F12 medium, and the cell density was 2×10⁶ cell/ml, ready for use.

### 1.2. Setting of Dosing Regimen

In this experiment, the animals were treated for three times with UV-oHSV2 stimulated mouse PBMC, and treated once every other two days, totally treated for three times, the detailed specific scheme is seen in the following Table 3.

TABLE 3
Dosing regimen of CT26 tumor model animal experiment

| | Animal number | | | | Injection |
|---|---|---|---|---|---|
| | in every | | | Volume | time |
| Groups | group | Sample | Sample concentration | (L) | (day) |
| Experiment | 3 | UV-oHSV2 | Cell density at 48 h | 100 | 0, 3, 6 |
| group | | stimulated | when stimulating | | |
| | | PBMC | PBMC by UV-oHSV2 | | |
| Blank | 3 | Physiological | — | 100 | 0, 3, 6 |
| control | | saline | | | |
| group | | | | | |

1.3. First Treatment of CT26 with UV-oHSV2 Stimulated Mouse PBMC
1) Preparation of blood sample: blood was collected from the eyeball of the mouse. First treatment: totally 5 ml of blood were collected from 7 mice. The preparation procedure of mouse PBMC was similar to Embodiment 1, to obtain a mouse PBMC cell suspension.
2) 1 ml of the obtained mouse PBMC cell suspension was separately taken and added into four wells of a 6-well plate, 40 μL of diabody (1:100) was added in every well, the final volume of every well was 4 ml, the experiment group was infected when MOI=1, the cell density was 2.0×10⁶ cell/ml, and a physiological saline was added in the blank control group. After the planking, the 6-well plate was put in the CO₂ incubator to be cultivated for 48 hours.
3) After 48 hours, the culture media in the holes were mixed, the bottom of the 6-well plate was gently blown and beaten, a small portion of adherent cell was blown and beaten down, the cells were counted, and in every group, 1 ml of the cells was taken ready for use in the animal experiment.
4) the tumor was induced and treated: 6 BALB/c normal mice were divided into 2 groups, 3 mice/group. 100 μl of liquid containing 2×10⁵ CT26 cells was subcutaneously injected at left latus of each of the 6 mice, as the tumor control in every group; in addition, 200 μl of UV-oHSV2 stimulated PBMC+CT26 cell mixed liquor (the mixing ratio was 1:1) was subcutaneously injected at right latus of each of the mice in the experiment group, and 200 μL of physiological saline+CT26 cell mixed liquor (the mixing ratio was 1:1) was injected subcutaneously at right latus of the mice in the blank control group.
   1.4. Second Treatment of CT26 with UV-oHSV2 Stimulated Mouse PBMC
1) Preparation of blood sample: blood was collected from the eyeball of the mouse. Second treatment: totally 6 ml of blood were collected from 11 mice. The preparation procedure of mouse PBMC was similar to the Embodiment 1, to obtain a mouse PBMC cell suspension.
2) 1 ml of the obtained mouse PBMC cell suspension was separately taken and added into four wells of the 6-well plate, and 40 μL of diabody (1:100) was added into every well, the final volume of every well was 4 ml, the experiment group was infected at MOI=1, and a physiological saline was added in the blank group. After the planking, the 6-well plate was put in the CO₂ incubator to be cultivated for 48 hours.
3) After 48 hours, the culture media in the holes were mixed, the bottom of the 6-well plate was gently blown and beaten, a small portion of adherent cell was blown and beaten down, the cells were counted, and in every group, 1 ml of the cells was taken ready for use in the animal experiment.
4) Second treatment of CT26 tumor: after the tumor in the mice was weighed, in the experiment group, 100 μL of UV-oHSV2 stimulated PBMC was in-situ injected intratumorally only at the right side of each of three mice with bilaterally induced tumor sites, and in the blank control group, 100 μL of physiological saline was injected intratumorally at the right side of each of three mice.
   1.5. Third Treatment of CT26 with UV-oHSV2 Stimulated Mouse PBMC

The experiment procedures were in accordance with ‘1.4 Second treatment of CT26 with UV-oHSV2 stimulated mouse PBMC’.

### 1.6. Observation

After finish of three times of treatment, the mice were observed and the tumor was weighed. The mice were observed twice every week until the end of the experiment. When the tumor disappeared, CT26 cells with higher cell density were in-situ injected, and the growing status of the tumor was observed.

### 1.7. Discussion on the Results

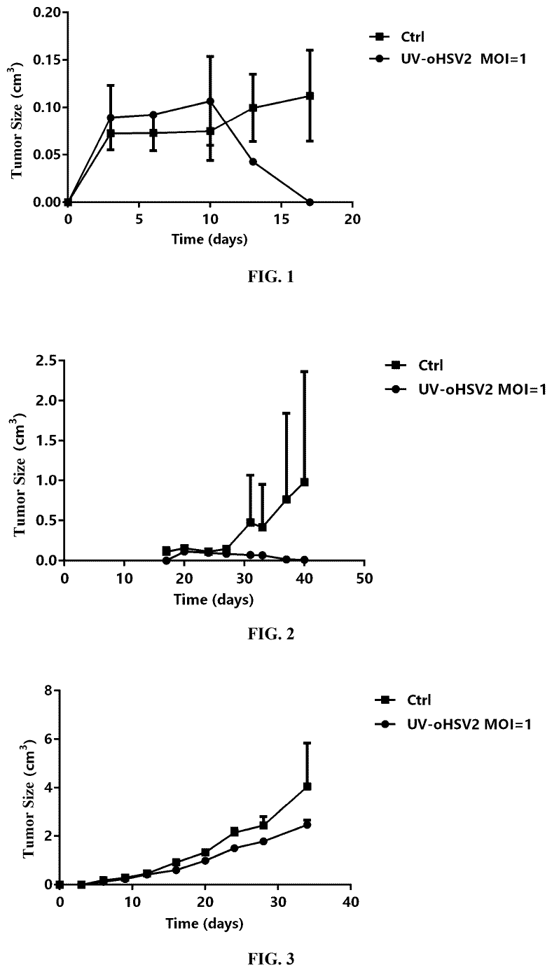

After three times of treatment on day 0, 3, 6 in the experiment, the mice were observed for 40 days, at 17th day of the observation, tumor in 3 mice all disappeared at the treatment site, whereas the mouse tumor size in the blank group always presented a trend of increase, as shown in FIG. 1.

On day 17, after the tumor disappeared, CT26 with a higher cell density (4×10⁶ cells/ml) was re-implanted, tumor implantation was conducted at the original tumor site, tumor was implanted at 100 μl/side for every mouse, the mice were continued to be observed, and observed twice every week. As shown in FIG. 2, with passage of time, it can be found that, when observed until day 40, the tumor in the blank control group presented a trend of continuous increase, whereas in the experiment group UV-oHSV2 (MOI=1) stimulated PBMC treatment group, at the tumor site of the second tumor implantation, a part of the tumor grew out, and a part of the tumor didn't grow out, after growth, a part of the tumor also disappeared with passage of time, suggesting that the UV-oHSV2 (MOI=1) stimulated PBMC treatment group might have a persistent immunological effect, and what immunocytes plays a role is under study.

### Experimental Example 2. In-Vivo Killing of 4T1 Tumor Cells by UV-oHSV2 Stimulated Mouse PBMC

This experimental example investigated the tumor treatment effect of UV-oHSV2 stimulated PBMC in-vivo against the mouse breast cancer 4T1. 4T1 originated from a BALB/c mouse breast cancer cell line has a 6-guanine resistance.

### 2.1. Preparation of 4T1 Cells

The mouse breast cancer cell (4T1) was cultivated according to the conventional method, and the medium was DMEM/F12 containing 10% fetal bovine serum. Before the tumor induction, the cells were collected, and centrifuged under 2800 rpm for 3 minutes, finally the cell precipitate was re-suspended with a serum-free DMEM/F12 medium, and the cell density was 4×10⁶ cell/ml, ready for use.

### 2.2. The Dosing Regimen is Seen in Table 4.

TABLE 4
Dosing regimen for 4T1 tumor model animal experiment

| | Animal number | | | | Injection |
|---|---|---|---|---|---|
| | in every | | | Volume | time |
| Groups | group | Sample | Sample concentration | (μL) | (day) |
| Experiment | 5 | UV-oHSV2 | Cell density at 48 | 100 | 0, 3, 6 |
| group | | stimulated | hours when | | |
| | | PBMC | stimulating PBMC | | |
| | | | by UV-oHSV2 | | |
| Blank | 5 | Physiological | — | 100 | 0, 3, 6 |
| control | | saline | | | |
| group | | | | | |

2.3. 4T1 was treated with UV-oHSV2 stimulated mouse PBMC for three times, and the experiment procedures were similar to those in the steps 1.3-1.5 of Experimental Example 1.

### 2.4. Observation

After finish of three times of treatment, the mice were observed and the tumors were weighed. The mice were observed twice every week until the experiment finished. When the tumor disappeared, 4T1 cells with higher cell density were in-situ injected, and the growing status of the tumors was observed.

### 2.5. Discussion on the Results

After three times of treatment on day 0, day 3, and day 6, an observation was conducted for 34 days, the tumor size at the treated side of the mouse is as shown in FIG. 3, it can be seen that the mouse tumor sizes in the control group and the UV-oHSV2 (MOI=1) stimulated PBMC treatment group all present a trend of increase, but the mouse tumor size in the UV-oHSV2 (MOI=1) stimulated PBMC treatment group increased more slowly (only a half of the size in the blank control group), there is no significant difference compared with the blank control group, but based on the observation from the tumor growth trend, the UV-oHSV2 (MOI=1) stimulation group has a certain inhibiting effect against growth of the tumor.

### Experimental Example 3. In-Vitro Killing of BGC823 Tumor Cells by UV-oHSV2 Stimulated Human PBMC

This experiment adopted human gastric adenocarcinoma cells (BGC823), and explored whether the PBMC after the proliferation activation had a killing effect on BGC823 cells by MTT colorimetry. The blood samples from four volunteers GQX, KZH, LBL, and SXT were used as the experiment subjects.

3.1 Planking of human PBMC, the experiment procedure was similar to the Embodiment 2.1, and the difference is in that, the set groups are shown in Table 5.

TABLE 5
Scheme of human PBMC planking

| | Groups (unit: μL) | | | | |
|---|---|---|---|---|---|
| | | | UV-oHSV2 | UV-oHSV2 | UV-oHSV2 |
| | Ctrl | PHA | MOI = 1 | MOI = 0.1 | MOI = 0.01 |
| Human PBMC | 500 | 500 | 500 | 500 | 500 |
| PHA | 0 | 5 | 0 | 0 | 0 |
| Inactivated virus | 0 | 0 | 200 | 20 | 2 |
| Inactivated plasma | 200 | 200 | 200 | 200 | 200 |
| 100 × diabody | 20 | 20 | 20 | 20 | 20 |
| DMEM/F12 | 1280 | 1275 | 1080 | 1260 | 1278 |

After the planking, a 6-well plate was put in the CO₂ incubator to be cultivated for 48 hours, after 48 hours, the culture media in the holes were mixed, the bottom of the 6-well plate was gently blown and beaten, a small portion of the adherent cells were blown and beaten down, the cells were counted with the cell counter, the cell densities in every group at 48 h were different, in order to unify the multiplicity of infection, the PBMC cell densities in every group were diluted to the same density, ready for use. That is, by ignoring the degree of PBMC proliferation in each group, the degree of PBMC activation in each group was analyzed at the same cell density.

### 3.2. Preparation of BGC823 Cells

BGC823 cells were cultivated by the conventional technique, the medium was removed after the culture, the cells were re-suspended and counted, and the cells were diluted to a cell density of 2×10⁵ cell/ml with DMEM/F12 containing 10% fetal bovine serum, ready for use.

### 3.3 BGC823 Cell In-Vitro Killing Planking

1) The experiment was divided into 5 groups, and every group had 5 holes, totally 150 μl/well of mixed liquor.
\textcircled{1} Experiment group one: 50 μl of UV-oHSV2 stimulated PBMC when MOI=1+50 μl of BGC823+50 μl of DMEM/F12 containing 10% FBS
\textcircled{2} Experiment group two: 50 μl of UV-oHSV2 stimulated PBMC when MOI=0.1+50 μl of BGC823+50 μl of DMEM/F12 containing 10% FBS.
\textcircled{3} Experiment group three: 50 μl of UV-oHSV2 stimulated PBMC when MOI=0.01+50 μl of BGC823+50 μl of DMEM/F12 containing 10% FBS.
\textcircled{4} Experiment group four: 50 μl of PHA (a positive stimulator) stimulated PBMC+50 μl of BGC823+50 μl of DMEM/F12 containing 10% FBS.
\textcircled{5} Blank group: 50 μl of BGC823+100 μl of DMEM/F12 containing 10% FBS.
2) After the planking, the 96-well plate was put in the CO₂ incubator to be cultivated for 48 to 72 hours.

### 3.4. MTT Detection

3.5. The experimental results are as shown in the following table.

TABLE 6
Table of results of in-vitro killing BGC823
in different groups for volunteer GQX

| | | | | | | Mean |
|---|---|---|---|---|---|---|
| Groups | OD value | | | | | value |
| UV-HSV2 (PBMC) | 0.870 | 0.905 | 0.949 | 0.865 | 0.994 | 0.917 |
| MOI = 0.01 | | | | | | |
| UV-HSV2 (PBMC) | 0.688 | 0.708 | 0.683 | 0.728 | 0.788 | 0.719 |
| MOI = 0.1 | | | | | | |
| UV-HSV2 (PBMC) | 0.586 | 0.561 | 0.537 | 0.596 | 0.554 | 0.567 |
| MOI = 1 | | | | | | |
| PHA(PBMC) | 0.935 | 0.950 | 0.925 | 0.931 | 0.923 | 0.933 |
| Ctrl | 0.932 | 0.938 | 0.896 | 0.936 | 1.011 | 0.943 |

TABLE 7
Table of results of in-vitro killing BGC823
in different groups for volunteer KZH

| | | | | | | Mean |
|---|---|---|---|---|---|---|
| Groups | OD value | | | | | value |
| UV-HSV2 (PBMC) | 0.853 | 0.928 | 0.939 | 0.952 | 0.981 | 0.9306 |
| MOI = 0.01 | | | | | | |
| UV-HSV2 (PBMC) | 0.828 | 0.93 | 0.957 | 0.92 | 0.916 | 0.9102 |
| MOI = 0.1 | | | | | | |
| UV-HSV2 (PBMC) | 0.934 | 0.847 | 0.841 | 0.923 | 0.932 | 0.8954 |
| MOI = 1 | | | | | | |
| PHA(PBMC) | 0.693 | 0.852 | 0.882 | 0.842 | 0.878 | 0.8294 |
| Ctrl | 0.933 | 1.001 | 0.967 | 1.005 | 1.051 | 0.9914 |

TABLE 8
Table of results of in-vitro killing BGC823
in different groups for volunteer LBL

| | | | | | | Mean |
|---|---|---|---|---|---|---|
| Groups | OD value | | | | | value |
| UV-HSV2 (PBMC) | 0.755 | 0.788 | 0.739 | 0.777 | 0.639 | 0.740 |
| MOI = 0.01 | | | | | | |
| UV-HSV2 (PBMC) | 0.706 | 0.740 | 0.661 | 0.685 | 0.678 | 0.694 |
| MOI = 0.1 | | | | | | |
| UV-HSV2 (PBMC) | 0.639 | 0.676 | 0.658 | 0.678 | 0.678 | 0.666 |
| MOI = 1 | | | | | | |
| PHA(PBMC) | 0.533 | 0.494 | 0.600 | 0.557 | 0.533 | 0.543 |
| Ctrl | 0.691 | 0.746 | 0.753 | 0.836 | 0.884 | 0.782 |

TABLE 9
Table of results of in-vitro killing BGC823
in different groups for volunteer SXT

| | | | | | | Mean |
|---|---|---|---|---|---|---|
| Groups | OD value | | | | | value |
| UV-HSV2 (PBMC) | 0.611 | 0.640 | 0.674 | 0.718 | 0.649 | 0.658 |
| MOI = 0.01 | | | | | | |
| UV-HSV2 (PBMC) | 0.603 | 0.616 | 0.636 | 0.627 | 0.655 | 0.627 |
| MOI = 0.1 | | | | | | |
| UV-HSV2 (PBMC) | 0.553 | 0.627 | 0.606 | 0.691 | 0.555 | 0.606 |
| MOI = 1 | | | | | | |
| PHA (PBMC) | 0.265 | 0.348 | 0.340 | 0.322 | 0.304 | 0.316 |
| Ctrl | 0.704 | 0.721 | 0.828 | 0.669 | 0.627 | 0.710 |

### 3.6. Discussion on Results

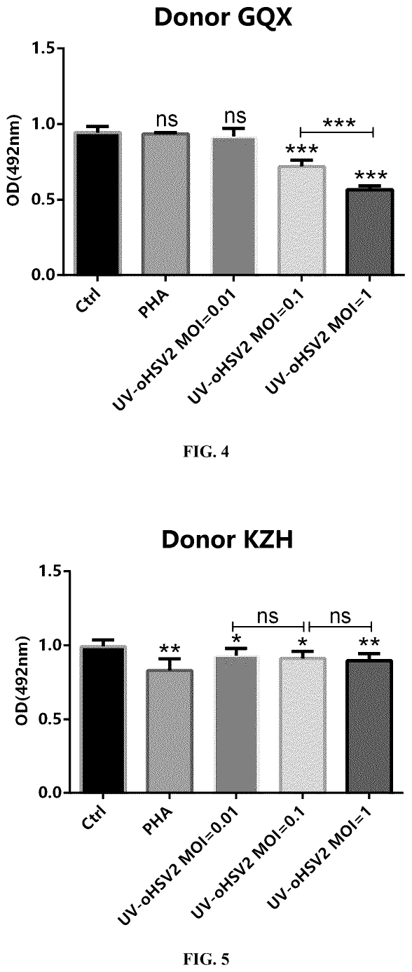

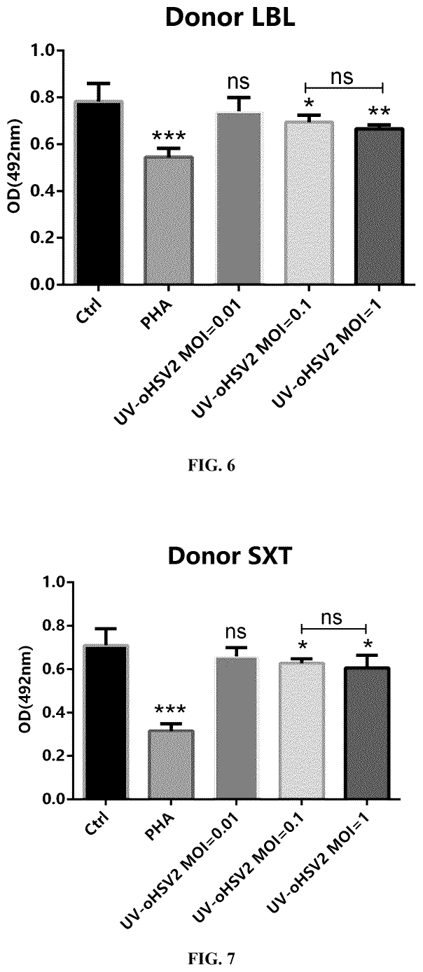

As shown in FIG. 4 to FIG. 7, from the experimental results of four different volunteers, it can be seen that, UV-oHSV2 stimulation group has a strong killing effect against BGC823 tumor cell when MOI-0.1 and MOI=1. And at high multiplicity of infection, the killing effect is higher. And in volunteer GQX, when MOI=1 the killing effect of UV-oHSV2 of the stimulated PBMC is better than that of the positive stimulator PHA.

### Experiment Example 4. In-Vivo Killing of LoVo Tumor Cells by UV-oHSV2 Stimulated Human PBMC

### 4.1. Preparation of LoVo Cells

The human colon cancer LoVo cells were cultivated according to the conventional method, and the medium was DMEM/F12 containing 10% fetal bovine serum. Before the tumor induction, the cells were collected, and centrifuged under 2800 rpm for 3 minutes, finally the cell precipitate was re-suspended with a serum-free DMEM/F12 medium, and the cell density was 1×10⁷ cell/ml, ready for use.

### 4.2. Setting of Dosing Regimen

This experiment adopted UV-oHSV2 stimulated Human PBMC treatment for three times, treated once every three days, totally treated for three times, the detailed specific scheme is seen in the following table.

TABLE 10
Dosing regimen of LoVo tumor model animal experiment

| | Animal number | | | | Injection |
|---|---|---|---|---|---|
| | in every | | | Volume | time |
| Groups | group | Sample | Sample concentration | (L) | (day) |
| UV-HSV2 | 3 | UV-oHSV2 | Cell density at 48 h when | 100 | 0, 3, 6 |
| (MOI = 0.01) | | (MOI = 0.01) | stimulating PBMC by | | |
| PBMC | | stimulated | UV-oHSV2 (MOI = 0.01) | | |
| | | PBMC | | | |
| UV-HSV2 | 3 | UV-oHSV2 | Cell density at 48 h when | 100 | 0, 3, 6 |
| (MOI = 0.1) | | (MOI = 0.1) | stimulating PBMC by | | |
| PBMC | | stimulated | UV-oHSV2 (MOI = 0.1) | | |
| | | PBMC | | | |
| UV-HSV2 | 3 | UV-oHSV2 | Cell density at 48 h when | 100 | 0, 3, 6 |
| (MOI = 1) | | (MOI = 1) | stimulating PBMC by | | |
| PBMC | | stimulated | UV-oHSV2 (MOI = 1) | | |
| | | PBMC | | | |
| PHA | 3 | PHA stimulated | Cell density at 48 h when | 100 | 0, 3, 6 |
| (PBMC) | | PBMC | stimulating PBMC by | | |
| PHA | | | | | |
| Ctrl | 3 | Blank stimulated | Cell density at 48 h when | 100 | 0, 3, 6 |
| (PBMC) | | PBMC | stimulating PBMC by | | |
| | | | blank | | |
| Blank | 3 | Physiological | — | 100 | 0, 3, 6 |
| control | | saline | | | |
| group | | | | | |

### 4.3 Treatment of LoVo by UV-oHSV2 Stimulated Human PBMC for Three Times

The experiment procedures were in accordance with the steps 1.3-1.5 of the Experimental Example 1, the difference is in that, the experiment mouse was BALB/c-nu nude mouse, the experiment groups were divided into three classes (they are respectively MOI=0.01/0.1/1), LoVo tumor implantation density: every mouse was injected with 100 μL of cell liquid containing 1×10⁶ LoVo.

### 4.4 Observation

After completion of three times of treatment, the mouse was observed, and the tumor was weighed. The mouse was observed twice every week until completion of the experiment. When the tumor disappeared, the LoVo cells with higher cell density were in-situ injected, and the growing status the tumor was observed.

### 4.5. Experimental Results

In the experiment, after three times of treatment on day 0, 3, 6, the mouse was observed for 38 days, as shown in FIG. 8, it can be found that, with passage of time, when the mouse was observed until day 38, the tumor in the blank control group presents a trend of continuous increase, whereas in the experiment group UV-oHSV2 (MOI=1) stimulated PBMC treatment group, an obvious inhibiting effect is achieved on tumor with passage of time (P=0.0035048). PHA, UV-oHSV2 (MOI=0.01/0.1) stimulated PBMC treatment groups likewise had an effect of inhibiting increase of the LoVo tumor cells (P values were respectively: P=0.026, P=0.0186, P=0.0196), and there is no statistical difference between the Ctrl stimulated PBMC treatment group and the blank control group (P=0.069>0.05).

### Experimental Example 5. Clinical Trials of VAK

### 2.6.2.2.4 Summary of Therapeutic Effects from IIT Clinical Trials

An Investigator-Initiated Clinical Trial (IIT) named “An Open Label, Randomized Controlled Clinical Study on the Safety and Efficacy of Virus Activated Killer Immune Cells (VAK) in the Treatment of Malignant Pleural and Peritoneal Effusion” is currently ongoing in China under the study identifier NCT05565014. It is a randomized, open-label, negative-controlled prospective study, aimed at evaluating the safety and efficacy of VAK cell therapy for malignant pleural and peritoneal effusion.

From July 2020 to May 2024, 6 patients with malignant pleural effusion were enrolled and received VAK treatment. All enrolled patients received systematic antitumor treatment during the trial.

The safety endpoint was evaluated via CTCAE 5.0 and efficacy endpoints (ORR_{effusion}/PFS_{effusion}, ORR) were evaluated via WHO 1979 and RECIST 1.1 criteria. The OS endpoint will be obtained by survival follow-up.

Below Table 11 summarizes the demographic data and efficacy outcomes for the malignant pleural effusion patients received VAK treatment.

TABLE 11
Summary of Demographic Data

| | VAK Treatment |
|---|---|
| | (n = 6) |
| Age (years) | |
| Mean (SD) | 58.5 (7.8) |
| Median | 58 |
| Min, Max | 45, 71 |
| Sex | |
| Male n(%) | 3 (50) |
| Female n(%) | 3 (50) |
| Total | 6 |
| Nationality | |
| Han n(%) | 6 (100) |
| Others n(%) | 0 (0) |
| Total | 6 |
| ECOG | |
| 0 | 0 (0) |
| 1 | 6 (100) |
| 2 | 0 (0) |
| Total | 6 |

Below Table 12 summarizes the overall efficacy data from per protocol set (PPS) for patients received VAK treatment.

TABLE 12
Summary of Efficacy Data

| | | VAK |
|---|---|---|
| | | Treatment |
| | | N = 6 |
| Overall ITT Efficacy Data | | n (%) |
| Per WHO 1979 | CR | 0 (0) |
| Criteria | PR | 3 (40) |
| | NC | 3 (60) |
| | PD | 0 (0) |
| ORR_{effusion} | | 50% |
| PFS_{effusion} (month) | | 8.41 |
| Per RECSIT | CR | 0 (0) |
| 1.1 Criteria | PR | 0 (0) |
| | SD | 6 (100) |
| | PD | 0 (0) |
| ORR | | 0% |
| mOS (month) | | 8.85 |

CR = complete response,
PR = partial response,
SD = stable disease,
PD = progressive disease,
NC = No Change
PFS = Progress Free Survival
mOS = Median Overall Survival
ORR = Objective response rate

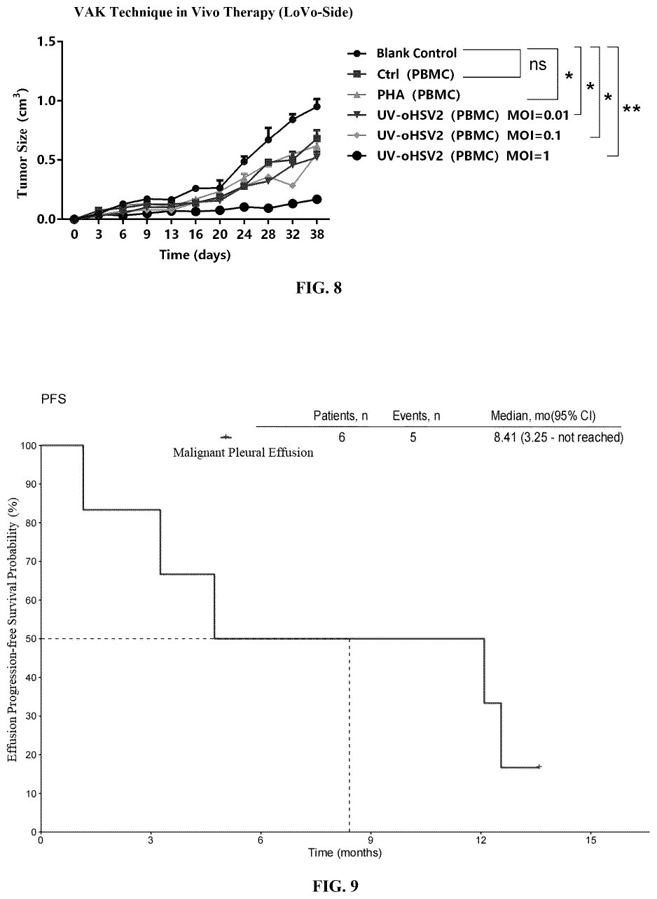

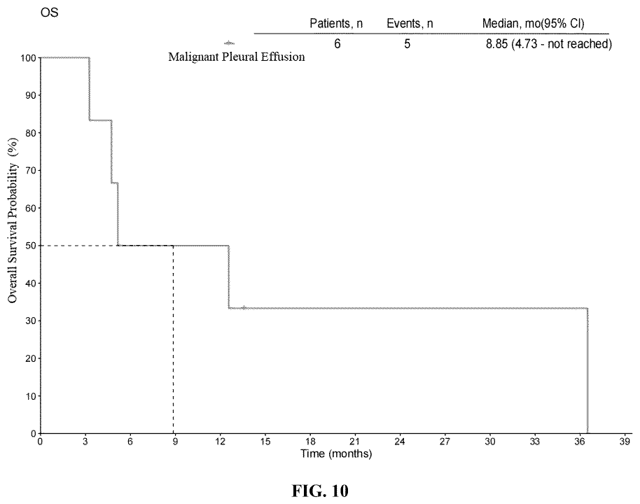

FIG. 9 shows Effusion progression free survival of MPE with VAK treatment. And FIG. 10 shows overall survival of MPE with VAK treatment.

Below Table 13 summarizes the effusion drainage volume data. (patients received other local treatment for MPE before VAK treatment)

TABLE 13
Summarize the effusion drainage volume data

| | Average Drainage Volume per day (mL) | | |
|---|---|---|---|
| Patient | Before VAK | Before VAK | After first |
| ID | treatment1 | treatment2 | VAK dosing |
| S001 | NA | NA | 85.7 |
| | | | (D 1-D 35) |
| S004 | NA | NA | 71.4 |
| | | | (D 1-D 14) |
| S007 | 150.0 | 246.7 | 180.3 |
| | About 4 months before | About 2 months before | (D 1-D 38) |
| 01-002 | NA | 216.7 | 118 |
| | | (D −6-D −1) | (D 1-D 20) |
| 01-003 | NA | 63.6 | 46.7 |
| | | About 1 month before | (D 1-D 6) |
| 01-006 | NA | 392.9 | 143.1 |
| | | (D −7-D −1) | (D 1-D 36) |

* NA = Not Applicable SD = stable disease

### Typical Cases: S001

TABLE 14
Evaluation of the Volume of Pleural Effusion:

| | Evaluation Period/Focus Value/Tumor Evaluation Conclusion | | | | | |
|---|---|---|---|---|---|---|
| | | Post- | | Follow-up | Follow-up | Follow-up |
| Subject | Screening | treatment | WHO tumor | tumor | tumor | tumor |
| number | period | evaluation | assessment | assessment 1 | assessment 2 | assessment 3 |
| S001 | 1194 mL | 893 mL | NE | 865 | 657 | 984 |
| | | Evaluation: SD | Evaluation: NE | Evaluation: SD | Evaluation: SD | Evaluation: SD |

* NE = inevaluable.,
SD = stable disease

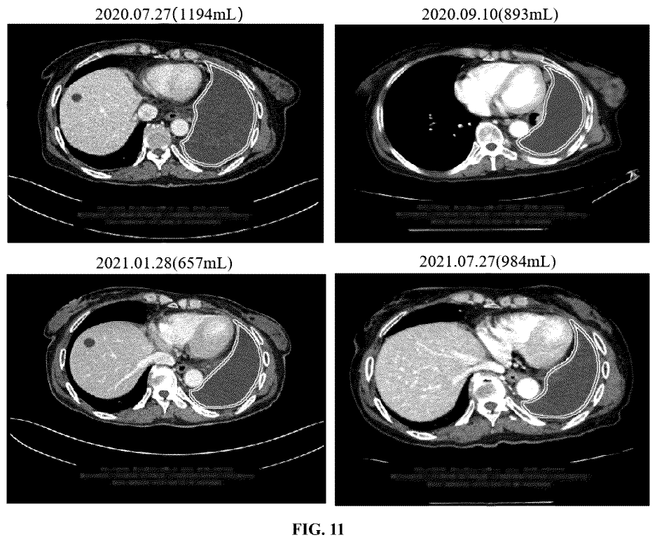

Patient S001 chest CT data and pleural effusion volume assessment table: pleural effusion volume decreased from 1194 mL (2020 Jul. 27) at baseline to 893 mL (2020 Sep. 10), 865 mL, 657 mL (2021 Jan. 28), 984 mL (2021 Jul. 27); as shown in FIG. 11.

TABLE 15
Target lesion Assessment:

| | Evaluation Period/Focus Value/Tumor Evaluation Conclusion | | | | |
|---|---|---|---|---|---|
| | | Initial | Follow-up | Follow-up | Follow-up |
| Subject | Screening | tumor | tumor | tumor | tumor |
| number | period | assessment | assessment 1 | assessment 2 | assessment 3 |
| S001 | Target | Target | 123.5 mm | 107.5 mm | 78.3 mm |
| | lesions: | lesions: | | | |
| | 147.7 mm | 94.1 mm | | | |
| | Non-target | Non-target | Non-target | Non-target | Non-target |
| | lesions: NE | lesions: Non- | lesions: Non- | lesions: Non- | lesions: non- |
| | | CR/non-PD | CR/non-PD | CR/n-PD | CR/n-PD |
| | New lesions: | New lesions: | New lesions: | New lesions: | New lesions: |
| | NE | None | None | None | None |
| | | Overall | Overall | Overall | Overall |
| | | Assessment: | Assessment: | Assessment: | Assessment: |
| | | PR | SD | SD | PR |

*NE = inevaluable.
,, SD = stable disease, PR = partial response,
Non-CR/non-PD′ is preferred over ‘stable disease’ for non-target disease since SD is increasingly used as endpoint for assessment of efficacy in some trials so to assign this category when no lesions can be measured is not advised.

Patient S001 Target Lesion Assessment Form: The total diameter decreased from 147.7 mm at baseline to 94.1 mm, 123.5 mm, 107.5 mm, and 78.3 mm respectively.

### Typical Cases: 01-002

TABLE 16
Evaluation of the Volume of Pleural Effusion:

| | Evaluation Period/Focus Value/Tumor Evaluation Conclusion | | |
|---|---|---|---|
| Subject | | Initial tumor | Follow-up tumor |
| number | Screening period | assessment | assessment 1 |
| 01-002 | Target lesions: 53 mm | Target lesions: 46 mm | Target lesions: 44 mm |
| | Non-target lesions: NE | Non-target lesions: | Non-target lesions: |
| | | Non-CR/non-PD | Non-CR/non-PD |
| | New lesions: NE | New lesions: None | New lesions: None |

NE = inevaluable.
Non-CR/non-PD′ is preferred over ‘stable disease’ for non-target disease since SD is increasingly used as endpoint for assessment of efficacy in some trials so to assign this category when no lesions can be measured is not advised.

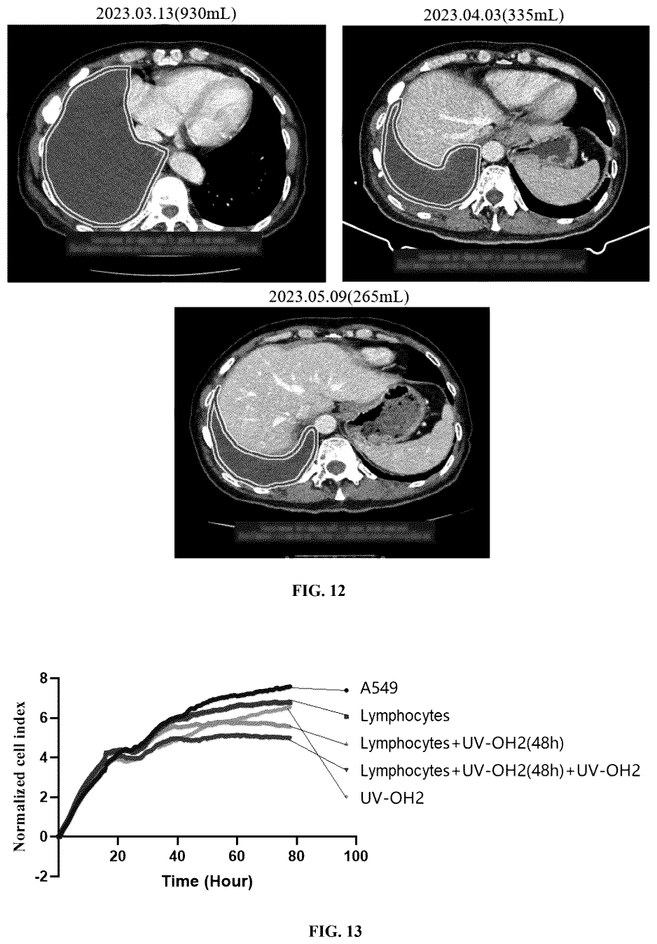

Patient 01-002 chest CT data and pleural effusion volume assessment table: The pleural effusion volume decreased from 930 mL at baseline to 335 mL (2023 Apr. 3) and 265 mL (2023 May, 09), respectively; as shown in FIG. 12.

Target lesion assessment: The total diameter decreased from 53 mm at baseline to 46 mm and 44 mm respectively.

VAK treatment has demonstrated preliminary efficacy in this study, effectively relieving the reaccumulating of malignant pleural, and prolonging effusion-progression free survival and overall survival of patients.

### Experimental Example 6. Effect Comparison of VAK (Activate Immune Cells Once VS Activate Immune Cells Twice)

Lymphocytes from the patient's pleural fluid were incubated with UV-OH2 (MOI=1) in vitro for 48 hours, and UV-OH2 was added to the lymphocytes again (MOI=1). Real-time cell analysis system was used to detect the real-time killing of A549 lung cancer cells by activated pleural fluid lymphocytes.

### 1. Preparation of A549 Cells

A549 cells were cultivated by the conventional technique, the medium was removed after the culture, the cells were re-suspended and counted, and the cells were diluted to a cell density of 2×10⁵ cell/ml, A549 cell suspension of 50 μl was placed into each well and cultured overnight in a CO₂ incubator for later use.

### 2. A549 Cells In-Vitro Killing Planking

1) Lymphocytes from the patient's pleural fluid were incubated with UV-OH2 (MOI=1) in vitro for 48 h and added to the plate of A549 cells cultured overnight according to the following groups.

The experiment was divided into 5 groups, totally 150 μl/well of mixed liquor.

Control Group 1:50 μl of A549+100 μl of 1640 containing 10% FBS (A549 Group)

Control Group 2:50 μl of A549+50 μL UV-OH2 (Dilute UV-OH2 with 1640 containing 10% FBS)+50 μl of 1640 containing 10% FBS (UV-OH2 Group).

Control Group 3:50 μl of Lymphocytes+50 μl of A549+50 μl of 1640 containing 10% FBS (This group of lymphocytes was incubated without UV-OH2 for 48 h and serve as a control; Lymphocytes Group)

Control Group 4:50 μl of UV-oHSV2 stimulated Lymphocytes when MOI=1+50 μl of A549+50 μl of 1640 containing 10% FBS. (Lymphocytes+UV-OH2 (48 h) Group)

Experiment group: 50 μl of UV-oHSV2 stimulated Lymphocytes when MOI=1+50 μl of A549+50 μl UV-OH2 (Dilute UV-OH2 with 1640 containing 10% FBS) (Lymphocytes+UV-OH2 (48 h)+UV-OH2 Group)
*1640 refers to the 1640 medium.
2) After the planking, the 16-well plate was put in the CO₂ incubator to be cultivated for 48 to 72 hours.

The process of lymphocyte separation is the same as Embodiment 3.

The preparation of lymphocyte in Control Group 3 was the same as steps 1)-12) of ‘(5) VAK Cell Production Process’ of Embodiment 3.

The lymphocyte in Control Group 4 were obtained by incubating the cells obtained from steps 1)-17) of ‘(5) VAK Cell Production Process’ of Embodiment 3 with UV-OH2 for 48 h.

As shown in FIG. 13, compared with Lymphocytes+UV-OH2 (48 h) group (Activate Immune Cells Once), Lymphocyte+UV-OH2 (48 h)+UV-OH2 group (Activate Immune Cells Twice) had stronger killing ability to A549 cells.

What is claimed is:

1. A method for preparing killer immunocytes, comprises the following steps:
(1) isolating immunocytes from malignant pleural effusion samples of a subject;
(2) co-incubating Ultra-Violet-inactivated herpes simplex virus type II with the immunocytes to activate the immunocytes;
(3) removing the Ultra-Violet-inactivated herpes simplex virus type II to obtain activated immunocytes;
(4) add new Ultra-Violet-inactivated herpes simplex virus type II to the activated immunocytes for secondary activation, thereby obtaining killer immunocytes for backinfusion into the subject's pleural fluid.

2. The method of claim 1, wherein, in step 2), a time of the co-incubating is 36-48 hour.

3. A method for treating lung cancer, comprising administering an effective amount of the killer immunocytes prepared by the method of claim 1 to a subject, and infusing more than 10⁶ killer immunocytes back into the subject's pleural fluid at a time.

4. The method of claim 3, wherein, a frequency of the infusing is once every 4-9 days, with 3-4 times per course of treatment, and the subject is given 1-2 courses of treatment.

5. The method of claim 3, wherein, a frequency of the infusing is once a week, with 3 times per course of treatment, and the subject is given 1-2 courses of treatment.
